(19) United States
(12) Patent Application Publication
(10) Pub. No.: US 2006/0148028 A1
(43) Pub. Date: Jul. 6, 2006

(54) Method for detecting microbe or cell

(76) Inventors: Naohiro Noda, Kanagawa (JP); Takuya Onodera, Kanagawa (JP); Koji Maruyama, Osaka (JP); Takeshi Saika, Osaka (JP); Yasunobu Tanaka, Osaka (JP); Masao Nasu, Osaka (JP); Nobuyasu Yamaguchi, Osaka (JP)

Correspondence Address: YOUNG & THOMPSON, 745 SOUTH 23RD STREET, 2ND FLOOR, ARLINGTON, VA, 22202, US

(21) Appl. No.: 10/526,985

(22) PCT Filed: Aug. 28, 2003
(86) PCT No.: PCT/JP03/10946

§ 371 Date: Oct. 28, 2005

(30) Foreign Application Priority Data
Sep. 5, 2002 (JP) 2002-259475

(51) Int. Cl. C12Q 1/04

(52) U.S. Cl. 435/34

(57) ABSTRACT

A method for detecting microorganisms or cells wherein microorganisms or cells contained in a sample are captured on the surface of the adhesive layer of a collection sheet having a substrate layer containing a focusing marker, an adhesive layer having a predetermined thickness and deposited on this substrate layer, the microorganisms or cells are stained by a staining reagent, and after being automatically focused, the light receiving optical system or the collection sheet is moved relatively by a distance equivalent to that obtained by adding the distance from the surface of the substrate layer to the position of the focusing marker to the predetermined thickness of the adhesive layer from the focusing position by this autofocusing reference point, the microorganisms or cells on the adhesive layer are automatically focused, and a light is radiated on the focused adhesive layer to measure the image and detect the microorganisms or cells.

### TECHNICAL FIELD

The present invention relates to a method of marking with a staining reagent microorganisms or cells contained in a sample and detecting the same by measuring their images. The microorganisms mentioned above include prokaryotes such as microbes, actinomycete and the like, eucaryotes such as yeast and mould, algae, viruses, and so forth. The cells include cultured cells derived from animals and plants as well as pollens of Japanese cryptomeria and Hinoki. The fields of application of the present detection method include medical treatment, manufacture of foodstuffs, water supply and drainage.

### BACKGROUND ART

The detection of microorganisms or cells of animals and plants in samples is industrially a very important art for, for example, for confirming sterilization and detecting any abnormality in the living condition of cells.

The method that has been generally used so far to detect bacteria and other microorganisms or cells existing on the surface to be examined and that cannot be observed by naked eyes is that of measuring by visually fixing with naked eyes or by a microscope for actual matters a colony that emerges by the culture method, or in other words by copying the microorganisms found on the surface to be examined on an agar medium realized in due form with agar by pressing the solid agar medium against the surface to be examined and by culturing the microorganisms in a suitable environment on the agar medium as they are. For example, the agar stump method based on the use of FOOD STAMP (made by Nissui Pharmaceutical Co., Ltd.) may be mentioned.

The membrane filter method based on the use of a membrane filter and the like capable of collecting microorganisms is a method of measuring a colony wherein microorganisms are collected while the surface to be examined are sufficiently wiped with saline, phosphate buffer and the like and the microorganisms are exposed, and after the microorganisms are collected on the membrane filter as this washed collector is filtered through a membrane filter, the microorganisms and the liquid culture medium are brought into a sufficiently close contact to form a colony on the filter. The membrane filter method can be used as a method for detecting microorganisms without culturing them by having the microorganisms captured on the filter come in contact with a suitable staining solution, and the number of colored cells is measured by a microscope and the like.

However, the agar stump method and the like in which normally an agar stump is used only once on a surface to be examined is inferior in terms of reproducibility due to a variable collection efficiency depending on the moisture ratio of the agar medium, and sometimes this causes inconveniences in the efficiency of collecting microorganisms. And a common problem for the culture method is that microorganisms are contaminated mutually and the impossibility of pure culture due to the reciprocal actions among microorganisms on the medium sometimes causes inconveniences in the subsequent judgments. And the application of the agar medium on the surface to be examined could contaminate the surface to be examined. In addition, there is a limitation in that the culture method is naturally limited to viable cells and involves a certain number of detections omitted. In addition, due to the necessity of providing one to two days or more for the period of culture, the culture method had an important limitation in that it was impossible to monitor microorganisms in real time.

And the membrane filter method had a disadvantage in that, although it is possible to filter a liquid subject of examination such as aqueous solution, non-liquid subjects of examination required tremendous human efforts to collect microorganisms including sampling by means of an applicator, the preparation of a washing liquid for exposing microorganisms and the like. In addition, the washing and filtering operation caused the matters collected other than microorganisms to swell and this obstructed the subsequent observations and measurements.

Lately, a microorganism testing method has been proposed to detect promptly and easily microorganisms on the surface of a solid body, wherein an adhesive layer mainly composed of water-soluble polymer of an adhesive sheet is pressed on the surface to be examined and peeled to collect the microorganisms found on the surface of the solid body, and then an aqueous solution containing one or more types of chromogenic substance or substances capable of staining microorganisms is made to get into contact with the surface of the adhesive layer, and the number of stained cells is observed and counted (image measurement) (see for example Japanese Patent Application Laid Open 10-70976).

However, these are cases of image measurement carried out by using a manually focused microscope or a similar image pickup device, and due to a narrow depth of field when used at a high magnifying power, it often takes much time to adjust the focus. Therefore, automatic focusing and automatic measurement have been desired.

With regard to a method of fluorescent image measurement involving the automatic focusing mentioned above, an application for the fluorescent image measurement method invented by some of the inventors of the present application was filed by Japanese Patent Application 2002-30648.

FIG. 1 shows an example of device for carrying out the method described in the Japanese Patent Application 2002-30648. According to the device shown in FIG. 1, the fluorescent image measurement method involving automatic focusing based on the image information obtained through an image pickup means includes radiating an auto-focusing (AF) light that illuminates in the wavelength band for fluorescent image measurement by the light source 2 from the same side as the radiation side of excitation beam as shown before irradiating the excitation beam to the sample 1 from the excitation beam source 10, judging the degree of focusing from the image information obtained thereby, driving at least one of sample 1 or the light receiving system in response to the degree to search the position of focus, stopping the radiation of the beam for AF upon reaching the position of focus, and then irradiating the excitation beam to the sample 1 from the light source 10. Thus, a fluorescent image measurement can be carried out. This device that uses no penetrating light enables to measure samples collected on the surface of the membrane filter.

As the light source 2 for AF, a light-emitting diode or a semiconductor laser is preferable. The image of samples during the radiation of AF beam is caught by an image pickup element 7 through an object lens 5, a dichroic mirror 3, a filter on the receiving side of fluorescent light 4 and an imaging lens 6. As the image pickup element, an element for CCD camera or an element for CMOS camera is preferable. Images obtained by the image pickup element 7 are sent to the calculating part 8, where the contrast is evaluated. For the evaluation of contrast, for example, the difference of luminance between adjacent pixels is calculated, and this is carried out by the general AF method wherein the position of the maximum contrast is considered as the position of focusing.

In FIG. 1, 9 represents a stage transfer mechanism, 11 represents a condenser of excitation beam, 12 represents a optical filter, and 13 represents a fluorescent light optical filter block. And although not shown in FIG. 1, for the evaluation of the contrast mentioned above, the Japanese Patent Application 2002-30648 discloses the method of marking on the surface of a slide glass for holding the sample, or marking on the surface of a membrane filter for filtering and collecting the sample (for details, see the Japanese Patent Application 2002-30648). According to the method mentioned above, the adoption of a simple mechanism with a limited number of element enables to avoid the optical quenching of the sample due to the radiation of the excitation beam making it impossible to detect the same and to autofocus (AF) samples collected on the surface of the membrane filter and samples with a weak contrast.

In the meanwhile, even the invention described in the Japanese Patent Application 2002-30648 described above has the following problem.

As the AF mark is provided on the surface of the slide glass for holding the sample or on the surface of the membrane filter for filtering and collecting the sample, the mark provided in the close vicinity of microorganisms and other samples constitutes an optical noise causing the precision of measurement to fall when an excitation beam is radiated to carry out a fluorescent image measurement.

In other words, in observing the image of microorganisms or cells, the mark on the surface of the membrane filter that holds the sample is reflected on the image, and constitutes a background noise that obstructs precise measurements. In particular, when observing faint light emitted by microorganisms or cells, the above-mentioned noise will be an important problem. For this reason, there has been a demand for a method of preventing marks on the sample holders (focusing marker) from being reflected and suppressing noises.

And the invention described in the Japanese Patent Application 2002-30648 mentioned above was mainly concerned with liquid samples and is based on the principle that microorganisms and the like existing on the surface of solid bodies are sampled with an applicator and the like as described above and are transformed into samples of liquid into which they are dispersed. Therefore, it is impossible to easily monitor in real time microorganisms existing on the surface of solid bodies and automatically focus and measure them.

The present invention has been realized by taking into consideration the above issues, and the object of the present invention is to provide a method of detecting microorganisms or cells capable of in particular easily monitoring in real time microorganisms existing on the surface of solid bodies and designed to improve the accuracy of automatic focusing and measurements.

### DISCLOSURE OF THE INVENTION

In order to resolve the issues mentioned above, the present invention includes the following steps in the process of marking microorganisms or cells contained in a sample (including the case of coexistence of both of them) with a staining reagent and detecting them by image measurement (invention of claim 1 hereof).

1) A step of capturing microorganisms or cells contained in the sample mentioned above on the adhesive layer of a collection sheet composed of a substrate layer having a focusing marker for autofocusing at least on its surface and an adhesive layer having a predetermined thickness deposited on the surface of this substrate layer.

2) A step of staining the microorganisms or cells captured as mentioned above with a staining reagent,

3) A step of autofocusing the focusing marker mentioned above,

4) A step of moving at least one of the light receiving optical system for image measurement or the collection sheet relatively by the equivalent distance to the predetermined thickness of the adhesive layer from the focusing position by the autofocusing mentioned above as a reference point, and focusing on the microorganisms or cells found on the adhesive layer mentioned above, and

5) A step of radiating light on the surface of the adhesive layer that had been brought into focus, measuring the images and detecting microorganisms or cells.

According to the detection method described above, it is possible to easily capture microorganisms existing on the surface of solid bodies on the adhesive layer mentioned above of the collection sheet. And due to the presence of an adhesive layer between the focusing marker on the surface of the substrate and microorganisms or cells, the focusing marker on the surface of the substrate does not create optical noises on the occasion of image measurements, and clear images of the microorganisms or cells captured can be obtained and therefore the microorganisms or cells can be measured with a high precision. Incidentally, the focusing marker on the surface of the substrate mentioned above may be formed by sandblasting, printing or other surface treatments or by creating optical patterns by mixing silica and other insoluble grains. The details of these operations will be described below.

The invention according to claim 1 above comprised capturing microorganisms or cells on the adhesive layer and then staining them. However, it is possible to stain them in advance of their capture as described below. In other words, the detecting process described in claim 1 above includes the following steps in place of the steps 1) and 2) described above (invention according to claim 2).

1) A step of staining in advance the microorganisms or cells contained in the sample by a staining reagent,

2) A step of capturing the microorganisms or cells contained in the sample that have been stained by the staining reagent in advance on the adhesive layer of a collection sheet composed of a substrate layer having at least on its surface a focusing marker for autofocusing and an adhesive layer having a predetermined thickness deposited on the surface of this substrate layer.

And in case where the fluorescence observation images of microorganisms or cells are obtained, as an embodiment of the invention according to claim 1 or claim 2, the invention according to claim 3 described below is preferable. In other words, in the detecting process according to claim 1 or 2, the fluorescent reagent mentioned above is chosen as a staining reagent and the excitation light is radiated on the adhesive layer mentioned above to carry out fluorescent image measurements, and in addition the radiation light for autofocusing on the occasion of autofocusing on the focusing marker mentioned above will be a light that includes a wavelength in the optical wavelength band for the fluorescent image measurement mentioned above (invention according to claim 3).

The meaning of choosing a light that includes a wavelength of the optical wavelength band for the fluorescent image measurement mentioned above as the radiation light for the autofocusing mentioned above is that it is intended to limit any focusing errors at the time of bringing the focusing marker into focus and bringing microorganisms or cells into focus.

In addition, in the detecting process according to any one of claims 1 to 3, the adhesive layer mentioned above comprises a non-water soluble adhesive (invention according to claim 4). This arrangement enables, when for example microorganisms or cells are marked with a fluorescent substance, to prevent the fluorescent substance from having difficulty in penetrating the adhesive layer, the adhesive layer from dissolving resulting in a movement of microorganisms or cells that have been captured, and the thickness and dimensions of the adhesive layer from changing thereby.

Furthermore, in the detecting process according to any one of claims 1 to 4 above, the predetermined thickness of the adhesive layer should be greater than the depth of field of the optical system (invention according to claim 5). This enables to measure in such a way that the focusing marker may not be reflected as the background noise during the observation of microorganisms or cells.

And as an embodiment different from the invention of the focusing marker mentioned above, it is possible to arrange things as described in the invention according to claim 6 described below. Specifically, in the detection process according to any one of claims 1 to 5, the focusing marker should be provided “on the back of or within the substrate layer” in place of “on the surface of the substrate layer”, and “the step of moving by a distance equivalent to the value of the predetermined thickness of the adhesive layer” in the step 4) above should be replaced by “the step of moving by a distance equal to the value of the distance obtained by adding the value of the distance from the surface of the substrate layer to the position of the focusing marker to the value of the predetermined thickness of the adhesive layer” (invention according to claim 6).

The focusing marker on the back of the substrate should be printed or treated by other means of surface treatment, and when it is in the substrate layer, it may be formed by the creation of an optical pattern by mixing silica and other insoluble grains. In the case of the invention according to claim 6, however, in particular in the optical passage from the surface of the adhesive layer to the focusing marker, because of the existence of two different materials of the adhesive layer and the substrate layer and a relatively greater value of optical distance, it is desirable to correct the distance of movement based on the refractive index of each of different materials as necessary. And with regard to the depth of field, all that is required to make the distance obtained by adding the value of distance from the surface of the substrate layer to the position of the focusing marker to the value of the predetermined thickness of the adhesive layer greater than the value of the depth of field of the optical system.

### BRIEF DESCRIPTION OF THE DRAWINGS

FIG. 1 is an illustration showing an example of configuration of a fluorescent image measuring device involving autofocusing described the Japanese Patent Application 2002-30648.

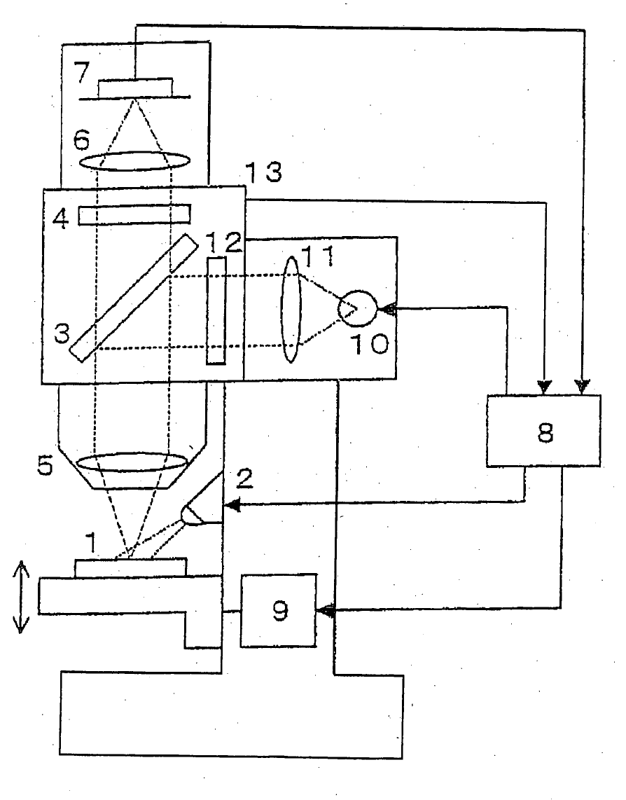

### DESCRIPTION OF SYMBOLS

1. Sample

2. Light source for autofocusing

3. Dichroic mirror

4. Optical filter on the receiving side of fluorescent light

5. Objective lens

6. Imaging lens

7. Image pickup element

8. Calculating part

9. Stage moving mechanism

10. Light source for excitation

11. Condenser lens for excitation beam

12. Optical filter

13. Fluorescent filter block

### THE BEST MODE FOR CARRYING OUT THE INVENTION

The collection sheet used in the present invention comprises an adhesive layer composed mainly by a polymer compound deposited on the substrate layer, and includes a focusing marker constituted by insoluble grains within or on the surface (including the interface between the substrate layer and the adhesive layer) or on the back of the substrate layer or a relief pattern on the surface of the substrate layer.

As a method of creating a focusing marker on the surface or the back of the substrate layer, the method of pressing to bring into relief or casting at the time of making the film for the substrate layer, the method of sandblasting or making similar treatments to the surface of the substrate layer, the method of printing on the surface of the substrate layer and the like can be mentioned. When the surface of the substrate layer is brought into relief by extruding the substrate layer at the time of forming the film for the substrate layer, casting, sandblasting and other treatments, the preferable depth of the relief is between 0.5 to 20 μm.

As for the printing method of the focusing marker, taking into consideration the fact that image contrast is judged at the time of focusing operation, flat all-over printing is not suitable and linear, checkered or dot patterns are preferable. And more preferably, at the time of obtaining images, a pattern of which at least a borderline is visible in the field of vision or with a changing color is desirable.

And when a focusing marker is provided within the substrate, the resin constituting film of the substrate layer may be made by mixing insoluble grains. For the insoluble grains, grains of calcium carbonate, titanium oxide, carbon black, silica, polystyrene, talc, asbestos, mica, clay, cellulose, starch and the like are indicated, and the grains having an average grain diameter of 0.5 to 20 μm are preferably used. These insoluble grains may be substituted by air bubble or carbon dioxide bubble.

Such a focusing marker may be disposed within the substrate layer, on the surface or on the back of the substrate layer of the collection sheet, and they may be doubled. For example, mixed insoluble grains of silica may be dispersed on the surface of the substrate layer to serve as the focusing marker on the surface of the substrate layer.

The adhesive layer mentioned above is not specially limited, provided that it has a sufficient power to capture microorganisms or cells on the surface to be examined and that it is a layer having a level surface structure of which the adhesive does not dissolve even if it is submerged in an aqueous solution for staining microorganisms or cells. However, when microorganisms or cells are marked with a fluorescent substance, in order to make it difficult for the fluorescent substance to penetrate into the adhesive layer, or in order to prevent the adhesive layer from dissolving causing the microorganisms or cells captured to move and also to prevent the value of thickness of the adhesive layer from changing, it is preferable to choose a non-water soluble adhesive for the principal component of the adhesive layer.

As the non-water soluble adhesive, for example acrylic adhesives, rubber adhesives and silicone adhesives may be used. And from the viewpoint of reducing the impacts on optical characteristics at the time of obtaining fluorescent images, for the substrate layer and the adhesive layer, the adoption of a highly transparent and non-fluorescent acrylic adhesive or silicone adhesive is preferable.

As acrylic adhesives, those mainly composed of, as monomer, an alkyl ester of (meth)acrylic acid such as ethyl (meth)acrylate, propyl (meth)acrylate, butyl (meth)acrylate, hexyl (meth)acrylate, octyl (meth)acrylate, 2-ethylhexyl (meth)acrylate, nonyl (meth)acrylate or decyl (meth)acrylate and, copolymerized therewith, one or more hydrophilic monomers such as (meth)acrylic acid, itaconic acid, maleic acid, hydroxyethyl (meth)acrylate, methoxyethyl (meth)acrylate, ethoxyethyl (meth)acrylate, butoxyethyl (meth)acrylate or ethyleneglycol (meth)acrylate can be mentioned. And it is preferable to crosslink such an adhesive layer by a treatment with a thermally crosslinking agent such as an isocyanate compound, an organic peroxide, an epoxy group-containing compound or a metal chelate compound or a treatment with ultraviolet rays, γ rays, electron beam or the like to enhance its adhesive characteristics.

As rubber adhesives, it is possible to use those comprising as the main polymer natural rubber, polyisobutylene, polyisoprene, polybutene, styrene-isoprene block copolymer, styrene-butadiene block polymer and the like and, incprporated therewith, a tackifier resin such as a rosin resin, a terpene resin, a coumarone-indene resin, terpene phenolic resin, a petroleum resin and so forth. As for silicone adhesive, an adhesive mainly composed of dimethylpolysiloxane may be exemplified.

From the viewpoint of adhesiveness on the surface to be examined, followability and collectability of microorganisms, it is preferable that the thickness of such an adhesive layer would be within a range of 5 to 100 μm. And at the time of obtaining the fluorescent images of the microorganisms or cells captured, it is preferable that the smoothness (distance from the top of convex to the bottom of concave) of the surface of the adhesive layer would be 20 μm or less. When roughness is 20 μm or less, the range of focusing of the fluorescent image-acquiring means will be extended, which will enable to treat images with a higher precision. Roughness can be determined by observing the section of the adhesive sheet with a surface roughness meter or an electronic microscope and by measuring the difference of altitude from the top of convexes to the bottom of concaves of the surface of the adhesive.

The material of the substrate of the collection sheet is not particularly limited as long as it is non-water soluble, does not form important ruggedness on the surface, and is a flexible material which can be freely fixed with pressure on a curved surface or a surface of sample set narrow space. Specifically, sheets, films, fabric, non-woven fabric, paper composed of polyester, polyethylene, polyurethane, polyvinyl chloride, and polyethylene laminate paper are indicated as examples. In particular, smooth sheets and films composed of polyester, polyethylene, polyvinyl chloride, polyurethane, etc. are preferable as the substrate. And the thickness of the substrate is not particularly limited as long as it is sufficiently strong as a supporting body, but a thickness approximately ranging from 5 to 200 μm is preferable.

The collection sheet used in the present invention can be produced by the methods already known. For example, it is produced by applying a solution containing a macromolecular compound used in the adhesive layer on the substrate and by drying the same at a temperature ranging from the room temperature to 200° C. In addition, the calendar method, the casting method and the extruding method can also be used. When a focusing marker is provided in the substrate, the substrate is produced by carrying out the surface treatment mentioned above or by adding insoluble grains, and it is preferable to provide the focusing marker on the substrate before depositing the adhesive layer. The sheet obtained thus can be cut in a freely chosen form for use.

According to the present invention, it is possible to sterilize the collection sheet by radiating radiant rays such as electron beams or γ rays and bridge at the same time the polymer compounds used in the adhesive layer. And it is also possible to sterilize the collection sheet by means of ethylene oxide and other similar gases, and to maintain the sterile condition by enclosing the collection sheet in a microorganisms insulating packing material in the sterilized condition.

For the purpose of the present invention, the term “microorganisms” includes, as described above, prokaryotes such as microbe and actinomycete, eucaryotes such as yeast and mould, algae, viruses and the like, and the term “cells” include cultured cells derived from animals and plants, pollens of Japanese cryptomeria and Hinoki.

According to the detection process of the present invention, it is possible to stain the microorganisms or cells that will be the subject of detection with one or more types of chromogenic substance or substances. The chromogenic substances are not particularly limited as long as they develop colors by reacting with the cell components contained in the microorganisms that are the subject of examination. However, as respresentative ones, it is possible to mention fluorescent stains that stain nucleic acid and protein. As specific chromogenic substances, in the case where microorganisms in general are the subjects, it is possible to mention fluorescent nucleic acid base analogs, fluorescent stain for staining nucleic acid, stain solution for staining protein, fluorescent probe used in the tissue analysis of protein, stain solution used in the analysis of cell membrane and membrane potential, stain solution used for marking fluorescent antibodies and the like, in the case where aerobic bacteria are the subjects, stain solutions and the like that develop color by the respiration of cells, in the case where eucaryotes are the subjects, a stain solution for staining mitochondria, a stain solution for staining Golgi bodies, a stain solution for staining endoplasmic reticula, in the case where the stain solution reacting with intracellular esterase and its modification compounds are directed towards advanced animal cells, a stain solution used for the observation of bone tissues, a stain solution serving as a nerve cell tracer and the like are mentioned, and microorganisms or cells stained by these stain solutions can be observed by means of a fluorescent microscope.

By choosing the type of these chromogenic substances, they can be applied to an extensive field including the measurement of the total number of cells for detecting all the microorganisms, the assay of staining and counting only microorganisms having a respiratory activity, the assay of staining and counting only microorganisms having an esterase activity, or the assay of staining and counting specific genera and species of microorganisms by using the double banding method combining a plurality of chromogenic substances and so forth.

In the present invention, the microorganisms or cells adhering to the surface to be examined are efficiently copied and captured by pressing the collection sheet on the surfaces to be examined including floor, walls, foodstuffs and the like. When the collection sheet is pressed on surfaces to be examined where there seem to be relatively few microorganisms or cells, a same surface of the collection sheet may be pressed a number of times. As the process of the present invention requires no culture as in the case of the agar stump method, there is no need to worry about the contamination of colonies and any possible changes in the phase of cells during their culture, and therefore it enables to capture microorganisms many times. Thus, many microorganisms or cells can be captured by increasing the number of pressing in the same way as filtering and condensing the microorganisms or cells that are dispersed in water by the membrane filter method.

Then, the collection sheets on which microorganisms or cells are collected are cut to a predetermined size as required, and the surface on which microorganisms or cells are collected is submerged in an aqueous solution containing a chromogenic substance to stain the microorganisms or cells. If it is necessary to remove excess chromogenic substance, the surface on which the microorganisms or cells are collected is washed and rinsed with sterilized water. And when it is necessary to dry the surface where the microorganisms or cells are collected after they have been stained, it is possible to dry them by air drying, natural drying or depressive desiccation.

Microorganisms or cells are counted and measured by obtaining their optical images by an optical microscope having an automatic focusing function, a fluorescent microscope, a laser microscope, a laser scanning site meter, or other suitable optical instruments and by measuring these images. The collection sheet of the present invention demonstrates its power and enables to measure images promptly. In other words, it is possible to focus the microorganisms or cells captured by focusing on the focusing marker in the collection sheet by taking advantage of the automatic focusing function and by shifting the focus by the thickness from the focusing position within the collection sheet to the surface of the adhesive layer. As this series of operations does not require cultivation, the microorganisms found on the adhesive surface of the collection sheet can be effectively detected within several minutes or ten and a few more minutes.

The present invention can be applied, for example, to environmental researches for measuring promptly the cleanness of the subjects of examination, because the collecting surface can be adhered to the surface to be examined, the microorganisms found on the surface for examination are copied, the microorganisms are stained without prior cultivation and the microorganisms can be observed at the single cell level. And it is possible and practical to capture microorganisms by applying the collection sheet several times on the surface to be examined and condense them, because they are captured at the single cell level. As fields of application, this method can be applied to the environmental study of microorganisms at the site of medical treatment and food processing.

The following is an example of embodiment wherein the fluorescent observation images of microorganisms or cells fluorescent stained by the method described above are obtained. Specifically, a reagent fluorescing by the esterase activity of the microorganisms or cells captured by the collection sheet, for example carboxy fluoresceine diacetate (hereinafter referred to as “CFDA”) is reacted. For obtaining the fluorescent images of the microorganisms or cells fluorescent stained by CFDA, an autofocusing light emitting the fluorescent wavelength light of CFDA (for example 500-550 nm) is radiated on the collection sheet. After focusing on the marker on the collection sheet, the distance between the detecting part of the optical system and the sample is moved from that position by a distance corresponding to the thickness between the focusing marker and the surface of the adhesive layer (for example 20 μm) and the focus is adjusted to the surface of the adhesive layer. A light having a wavelength capable of exciting CFDA (for example 450-500 nm) is radiated on the focused collection sheet to obtain the fluorescent image of the surface of the adhesive layer. And from the fluorescent images obtained there, microorganisms or cells are recognized and detected.

### EMBODIMENT

The present invention will be described more specifically below by referring to a plurality of embodiments. However, these are merely examples and do not limit in any way the scope of the present invention.

### Embodiment 1

1) Fabrication of a Collection Sheet

A copolymer toluene solution with a gel ratio of 40% (w/w) is obtained by polymerizing isononylacrylate, 2-methoxyethylacrylate and acrylic acid (65/30/5 input weight ratio) using azoisobutyronitrile as a polymerization initiator. This solution is applied on a film of which a 25 μm thick transparent polyester non-adhesive surface is scratched to a depth of approximately 1 μm by a #1200 sandpaper so that the thickness may be 20 μm when dry and a polyester film 26 μm thick to which powdered silica with an average grain diameter of 5 μm are mixed, and these films are dried for five (5) minutes at 130° C. And then these films are sterilized with γ beam having a dosage of 25 k grey to produce a collection sheet. In the meanwhile, the case of using the powdered silica as the focusing marker will constitute the embodiment 1-1 described further below, and the case of using the treatment of the surface of the substrate with a sandpaper as the focusing marker will constitute the embodiment 1-2 described further below.

2) Capture and Staining of Microorganisms

0.1 mL of solution obtained by diluting 100 times with sterilized water the culture medium of Staphylococcus epidermidis IFO3762 is filtered through a polycarbonate membrane having straight holes with a diameter of 0.4 μm and the microorganisms found on the flat membrane and washed by a sterilized phosphate buffer are taken as the subjects of examination, and the collection sheet produced by the step 1) above is pressed on the filter surface and is peeled. Then, a phosphate buffer containing 0.1% of 6-carboxy fluoresceine diacetate is dripped as a stain solution to the surface where the microorganisms are collected. After being left unattended for three (3) minutes at the room temperature and stained, the surface where the microorganisms are captured is again washed with a phosphate buffer.

3) Counting

The sample images are obtained by means of an optical system provided with a CCD camera as an image pickup device having a multiplication of 10 times. This image information served as the basis of driving as least one of the mirror barrel of the light receiving system or the sample stage by the personal computer and searching the focusing position. For this driving, it is suitable to use a stepping motor capable of controlling positions with a resolving power of approximately 0.5-1 μm. By preparing an optical device having such mechanism (hereinafter referred to as “the measuring device”), the number of microorganisms is counted on the surface where microorganisms are collected on the collection sheet wherein the collected microorganisms are stained.

Specifically, in the first place, at least either one of the optical system tube or the sample stage is moved in the direction of separating from the vicinity of the substrate, and the focus point where the focusing marker in the form of powdered silica and the like indicates the focus image obtained is stored. After further moving over a predetermined distance, for example 20 μm, until the point where the focusing is completed on the surface of the adhesive layer, the sample image is obtained. In the case of fluorescence observation, it is possible to identify the microorganisms or cells as luminescent spots in the fluorescent image by radiating an excitation beam having a predetermined wavelength.

And it is possible to measure without causing the focusing marker to be reflected as the background noises at the time of observing microorganisms or cells by making the value of the distance between the focusing marker and the surface of the adhesive layer greater than the depth of field of the optical system. The depth of field depends on the aperture of the optical system, and in normal microscopic observation, it is several μm. As a result, it is possible to prevent the focusing marker from being reflected on the image obtained and constituting the background noises by setting the distance between the focusing marker and the surface of the adhesive layer at 20 μm.

The number of microorganisms or cells found in the field of vision can be counted by measuring the image obtained. And it is possible to reduce statistical variation and to measure more accurately by driving either one of the mirror barrel or the sample stage, observing different positions on the sample and counting the number of microorganisms or cells in a plurality of fields of vision. In the present embodiment, the sample stage was driven, the images of a total of 70 fields of vision were obtained and the number of bacteria contained there was counted. And a sterilized solution was chosen as the subject of examination in place of a diluted culture broth, and the adhesive surfaces of collection sheets on which microorganisms are not captured were also measured in the same way.

4) Data Analysis

The same samples as those used in the measurement mentioned above were measured by the culture method to compare with the measured value of number of bacteria by the present invention. The number of bacteria measured by the culture method totaled 3,028/mm². The measurement result of the culture method was compared with the measurement value for the number of cells based on the present invention that served as the reference, in other words by the recovery ratio of bacteria.

And the same value was compared with the case of no focusing marker being used (Comparative Example 1) described below.

Comparative Example 1

The collection sheet was produced in the same way as the Embodiment 1 except that a transparent polyester film 25 μm thick of which no treatment was made on the substrate, and microorganisms were captured, stained and washed.

Both the measurement result of Embodiments and the Comparative Example 1 are shown in Table 1. Incidentally, in the remark column of Table 1, as stated above, the case of using powdered silica as the focusing marker is shown as the Embodiment 1-1, the case of using the sandpaper treatment of the substrate surface as the focusing marker is shown as the Embodiment 1-2, and the cases marked with a suffix “a” show cases where no microorganism was available for examination. The same thing applies to the Comparative Example 1.

TABLE 1

| | | Number of | Recovery | |
|---|---|---|---|---|
| Focusing | Microorganisms | bacteria | ratio of | |
| marker | examined | measured | bacteria | Remarks |
| Powdered | S. epidermidis | 3,149/mm² | 104% | Embodiment |
| silica | | | | 1-1 |
| (in the | | | | |
| substrate) | | | | |
| Powdered | None | 29/mm² | 1% | Embodiment |
| silica | | | | 1-1a |
| (in the | | | | |
| substrate) | | | | |
| Papersand | S. epidermidis | 2,846/mm² | 94% | Embodiment |
| treatment | | | | 1-2 |
| on the | | | | |
| substrate | | | | |
| Papersand | None | 12/mm² | <1% | Embodiment |
| treatment | | | | 1-2a |
| on the | | | | |
| substrate | | | | |
| None | S. epidermidis | 0/mm² | 0% | Comparative |
| | | | | Example 1 |
| None | None | Unmeasurable | — | Comparative |
| | | (no focusing) | | Example 1a |

As Table 1 shows clearly, the automatic focusing function worked on the focusing marker of the collection sheet and S. epidermidis could be measured in the Embodiment 1-1 and the Embodiment 1-2. The reason why the collection sheets that do not capture at all microorganisms (Embodiment 1-1a and Embodiment 1-2a) detect a small number of microorganisms is that the microorganisms and fluorescent grain noises in the environment of measurement may have slipped in and that it may be affected by mistake in the image processing. The measurements of 3,149/mm² and 2,846/mm² in Table 1 seem to include errors of a similar magnitude.

In the case of Comparative Example 1 in which no focusing marker was provided, no focusing was completed and no measurement could be carried out. Even if no focusing marker is available, the samples themselves (for example, S. epidermidis captured) are often mistaken as a pseudo-focusing marker, which can be subjects of automatic focusing. In such a case, however, a sample image is obtained at a position forcibly shifted further from the focusing position by a predetermined distance (for example, 20 μm), microorganisms cannot be accurately focused and the luminance spots derived from the microorganisms cannot be identified in the image. When no focusing marker is provided in the collection sheet like this, no appropriate focusing can be made on samples, and therefore it has become clear that it is incomplete as a measurement system.

### Embodiment 2

A procedure similar to that of the Embodiment 1 was considered except that the microorganism to be tested is Escherichia coli K-12 and that a collection sheet including a substrate in which silica is mixed is adopted. The results will be shown in Table 2 along with the Comparative Example 2.

(Control 2)

The collection sheet was produced in the same way as the Embodiment 2 except that a transparent polyester film 25 μm thick of which no treatment was made on the substrate, and microorganisms were captured, stained and washed.

TABLE 2

| | Micro- | Number of | Recovery | |
|---|---|---|---|---|
| Focusing | organisms | bacteria | ratio of | |
| marker | examined | measured | bacteria | Remarks |
| Powdered | E. coli | 2,147/mm² | 62% | Embodiment 2 |
| silica | K-12 | | | |
| (in the | | | | |
| substrate) | | | | |
| None | E. coli | 0/mm² | 0% | Comparative |
| | K-12 | | | Example 2 |

In the Embodiment 2, the automatic focusing function works on the focusing marker of the collection sheet, and the number of E. coli K-12 bacteria could be measured. However, the recovery ratio of bacteria varies depending on the type of bacteria due to differences in stainability by the reagent (in the Embodiment 2 above, 6-carboxy fluoresceine diacetate) in addition to the impact of the sample condition and depending on the microorganism. In the case of S. epidermidis of the Embodiment 1 mentioned above, it had a value close to approximately 100%. However, in the case of E. coli K-12 of the Embodiment 2 and the case of E. coli O157 described below, it was approximately 60%. In this case, the measurement value of the present invention can be converted by the recovery rate of bacteria to be the real value.

In the case of the Comparative Example 2 without any focusing marker, focusing could not be completed and no measurement could be carried out. Therefore, when no focusing marker is provided in the collection sheet, the whole system is unsuitable as a measuring system because it is impossible to focus.

### Embodiment 3

The microorganism E. coli O157 was chosen as the microorganism to be tested, and this embodiment was examined in the same way as the Embodiment 2. However, with regard to staining, FITC labeled antibody E. coli O157 antibody (made by KPL Inc., diluted by phosphate buffer saline so as to be 0.05 mg/ml) was chosen for the bacteria, and after five (5) minutes of staining, the bacteria were washed with sterilized water. The results are shown in Table 3 along with the following Comparative Example 3.

### Comparative Example 3

The collection sheet was produced in the same way as the Embodiment 3 except that a transparent polyester film 25 μm thick of which no treatment was made on the substrate, and microorganisms were captured, stained and washed.

TABLE 3

| | Micro- | Number of | Recovery | |
|---|---|---|---|---|
| Focusing | organisms | bacteria | ratio of | |
| marker | examined | measured | bacteria | Remarks |
| Powdered | E. coli | 2,186/mm² | 60% | Embodiment 3 |
| silica | O157 | | | |
| (in the | | | | |
| substrate) | | | | |
| None | E. coli | 0/mm² | 0% | Comparative |
| | O157 | | | Example 3 |

In the Embodiment 3, the automatic focusing function works on the focusing marker of the colleting sheet, and the number of E. coli O157 bacteria could be measured. Although the staining mechanism of microorganisms is different from that of the Embodiment 1 and the Embodiment 2, there was absolutely no inconvenience in detection.

In the case of the Comparative Example 3 without any focusing marker, no focusing could be carried out and it was impossible to measure.

### Embodiment 4

The culture broth E. coli K-12 was stained, measured, and the time required for counting a freely chosen number of bacteria was measured according to the method described in the Embodiment 2. The results are shown in Table 4 along with the Comparative Example 4 shown below.

### Comparative Example 4

The culture broth E. coli K-12 was diluted as required by a phosphate buffer and 6-carboxy fluoresceine diacetate was added thereto so that its final density may be 0.1%, and a staining was carried out for three (3) minutes at the room temperature. This solution was filtered through a polycarbonate membrane to collect the bacteria. The membrane that collected the bacteria was observed by a fluorescent microscope under a blue excitation beam at a multiplication of 400 times, and the number of fluorescent cells was counted.

TABLE 4

| | Micro- | Number of | | |
|---|---|---|---|---|
| Measuring | organism | bacteria | Time required | |
| method | tested | counted | for counting | Remarks |
| Present | E. coli | 20,000 | 10 minutes | Embodiment 4 |
| invention | K-12 | or more | | |
| Observation | E. coli | About | 45 minutes | Comparative |
| by fluorescent | K-12 | 3,000 | | Example 4 |
| microscope, | | | | |
| visual | | | | |
| counting | | | | |

The detection method of the present invention required only 10 minutes to analyze 20,000 or more bacteria.

The method used in the Comparative Example 4, on the other hand, required 45 minutes to count approximately 3,000 bacteria. This is not only attributable to the trouble of counting by man power but also to the necessity of changing the field of vision of the fluorescent microscope in the process of counting and moreover to the time required for the operation of focusing each time.

The results shown in Table 4 demonstrates that the method of the present invention is effective for promptly and easily detecting microorganisms or cells.

### INDUSTRIAL APPLICABILITY

As described above, according to the present invention, the microorganisms or cells contained in samples are captured on the surface of the adhesive layer of a collection sheet comprising a substrate layer containing on the surface, the back or within the substrate a focusing marker for autofocusing, an adhesive layer having a predetermined thickness and deposited on the surface of this substrate layer, the microorganisms or cells are stained by a staining reagent before or after their capture, and after being automatically brought into focus by the focusing marker, at least either one of the light receiving optical system for image measurement or the collection sheet is moved relatively by a distance equivalent to the distance obtained by adding the value of distance from the surface of the substrate layer to the position of the focusing marker to the value of the predetermined thickness of the adhesive layer (if the focusing marker is provided on the surface of the substrate layer, the added value is zero) from the focusing position by this autofocusing as the reference point, the microorganisms or cells on the adhesive layer are brought into focus, and a light is radiated on the surface of the focused adhesive layer to image measure and detect the microorganisms or cells. Therefore, the present invention enables in particular to monitor easily and in real time microorganisms existing on the surface of solid bodies, and in addition provides a method of detecting microorganisms or cells with an improved accuracy of measuring automatic focusing.

1. A method of marking by a staining agent microorganisms or cells contained in a sample and detecting the same by measuring image, comprising;
1) a step of capturing said microorganisms or cells contained in said sample on the adhesive layer of a collection sheet composed of a substrate layer having a focusing marker for autofocusing at least on its surface and an adhesive layer having a predetermined thickness deposited on the surface of this substrate layer,
2) A step of staining said captured microorganisms or cells by a staining reagent,
3) A step of autofocusing said focusing marker
4) A step of moving at least one of the light receiving optical system for image measurement or the collection sheet relatively by the equivalent distance to the value of said predetermined thickness of the adhesive layer from the focusing position by said autofocusing as a reference point to bring said microorganisms or cells on the adhesive layer into focus, and
5) A step of radiating light on the surface of said adhesive layer that had been brought into focus and detecting the microorganisms or cells by measuring image.

2. The method of detecting microorganisms or cells according to claim 1 comprising the following steps in place of said steps 1) and 2).
1) A step of staining said microorganisms or cells contained in said sample in advance by a staining reagent, and
2) A step of capturing said microorganisms or cells contained in the sample stained in advance by a staining reagent on said adhesive layer of a collection sheet comprising a substrate layer having a focusing marker for autofocusing at least on its surface and an adhesive layer having a predetermined thickness deposited on the surface of this substrate layer.

3. The method of detecting microorganisms or cells according to claim 1, wherein said staining reagent is a fluorescent reagent, an excitation light is radiated onto the surface of said adhesive layer to measure fluorescent image, and the radiation light for autofocusing when said focusing marker is automatically brought into focus is a light that includes a wavelength of the optical wavelength band for said fluorescent light image measuring.

4. The method of detecting microorganisms or cells according to claim 1, wherein said adhesive layer comprises a non-water soluble adhesive.

5. The method of detecting microorganisms or cells according to claim 1, wherein the value of the predetermined thickness of said adhesive layer is greater than the depth of field of the optical system.

6. The method of detecting microorganisms or cells according to claim 1, wherein said focusing marker is provided “on the back of the substrate layer or within the substrate layer” in place of said “on the surface of the substrate layer,” and “moving by a distance equivalent to the distance obtained by adding the value of distance from the surface of the substrate layer to the position of the focusing marker to the value of the predetermined thickness of the adhesive layer” in place of “moving by an equivalent distance to the value of said predetermined thickness of the adhesive layer” in said step 4).

7. The method of detecting microorganisms or cells according to claim 2, wherein said staining reagent is a fluorescent reagent, an excitation light is radiated onto the surface of said adhesive layer to measure fluorescent image, and the radiation light for autofocusing when said focusing marker is automatically brought into focus is a light that includes a wavelength of the optical wavelength band for said fluorescent light image measuring.
